(19) United States
(12) Patent Application Publication
(10) Pub. No.: US 2006/0142588 A1
(43) Pub. Date: Jun. 29, 2006

(54) Acetoacetylation of alcohols, thiols and amines in a microreactor

(76) Inventors: Uwe Nickel, Bad Homburg (DE); Detlef Wehle, Brechen (DE); Leonhard Unverdorben, Nidderau (DE); Klaus Forstinger, Babenhausen (DE); Hieng Kim, Nidderau (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/544,076

(22) PCT Filed: Dec. 13, 2003
(86) PCT No.: PCT/EP03/14200

§ 371 Date: Jul. 29, 2005

(30) Foreign Application Priority Data
Jan. 30, 2003 (DE) 103 03 581.8

(51) Int. Cl. C07D 235/22; C07C 69/76; C07C 309/29

(52) U.S. Cl. 548/307.7; 560/10; 560/55

(57) ABSTRACT

The invention relates to a method for the production of β-keto-carboxylic acid derivatives of formula (I) or the salts thereof, where X═NR′, O or S, R, R′, R¹-R⁴ independently ═H, alkyl, alkenyl, aryl or heteroaryl, by reaction of a diketene of formula (II) with a compound comprising an active hydrogen of formula ROH, NHRR′ or RSH, characterised in that the reaction is continuously carried out in a microreactor.

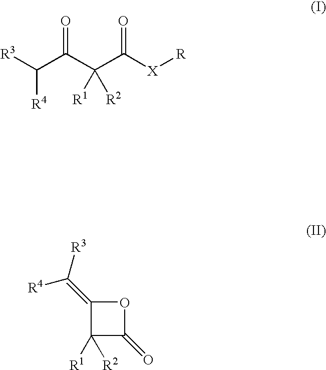

The present invention relates to an environmentally friendly, economically viable process, which is very safe with regard to the risk potential, for acetoacetylating alcohols, thiols and amines with diketene and derivatized diketenes.

The products of the acetoacetylation of alcohols, amines and thiols are important intermediates in numerous chemical reactions. For example, the acetoacetylation of alcohols leads to the product group of the acetoacetyl esters, which constitute important raw materials for the preparation of intermediates for the synthesis of active pharmaceutical ingredients, paints and agrochemicals. The acetoacetylation of amines gives rise to the product group of the acetoacetylamides which are versatile intermediates, for example for the preparation of pigments and of reactive dyes. Industrially, the products are prepared conventionally in a batch process. In addition, continuous processes in thin-film evaporators, tanks, mixers and loop reactors are also described.

For the purity, quality and consistent quality of the products, the monitoring of the process parameters, such as temperature, time and mixing, is essential in all processes. In the scale-up of new products from the laboratory scale to the industrial production scale, particularly in the case of batch processes, lies a further, very substantial difficulty. In particular, the prior art processes, even when the process conditions are monitored very carefully and the reaction is controlled strictly, lead to impurities.

DE-A-26 12 391 relates to a continuous process for preparing 5-acetoacetylaminobenzimidazolone from 5-aminobenzimidazolone-2 and diketene in water.

EP-A-0 648 748 relates to a process for preparing 5-acetoacetylamino-benzimidazolone-2 from 5-aminobenzimidazolone-2 and diketene in the presence of a water-soluble (C₁-C₄)-alcohol or of a mixture of this alcohol in water. In Example 1, a solution which contains 10.6 percent by weight of 5-aminobenzimidazol-2-one which has been prepared at 82° C. in a solvent mixture of in each case 50 percent by weight of water and ethanol is reacted with diketene continuously in a reactor with stirrer, thermometer and bottom outlet at 85° C. The amine solution and excess diketene are metered in simultaneously. The use of a solvent mixture and the high temperatures are disadvantageous, since diketene forms, under these reaction conditions, both with water and with the alcohols, undesired by-products which have to be removed from the desired reaction product, 5-acetoacetylaminobenzimidazol-2-one, and lead to a higher consumption of diketene. The high dissolution capacity of the solvent mixtures of water and of a (C₁-C₄)-alcohol has a particularly disadvantageous effect on the products, so that a large amount of energy has to be applied for cooling to 15° C. to crystallize the products. To increase the yields, the mother liquor additionally has to be recycled into the production process.

It is therefore an object of the present invention to find a process for aceto-acetylating alcohols, thiols and amines, in which the process parameters can be controlled optimally and pure reaction products are formed with a minimum level of by-products which are difficult to remove and/or unconverted starting products, and which enables the simple scale-up from the laboratory scale to the industrial scale.

It is known that certain chemical reactions can be performed in micro-reactors. Microreactors are constructed from stacks of structured plates and are described, for example, in DE-A-30 26 466. It is also known that microreactors are utilized for reactions which do not require or produce any materials or solids which can block the microchannels.

It has now been found that microreactors are surprisingly suitable for the acetoacetylation of alcohols, thiols and amines. The products are obtained under the selective reaction conditions as liquid products, melts, dissolved or crystallized. The use of microreactors not only allows the ratio of diketene to amine, alcohol or thiol to be distinctly reduced compared to the prior art, but distinctly reduced concentrations of by-products surprisingly also occur. Furthermore, a reaction in water without the use of solvent mixtures is possible, as a result of which the removal of possibly combustible solvents for the isolation of the end product is dispensed with. The reaction products may be used without further purification and isolation steps.

The invention provides a process for preparing β-keto carboxylic acid derivatives of the formula (I) or salts thereof 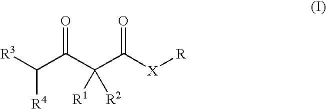
where
X is NR′, O or S;
R, R′ are each independently H, straight-chain, branched or cyclic alkyl or alkenyl having from 1 to 18 carbon atoms, aryl or heteroaryl, in which one or more hydrogens in the said alkyl, alkenyl, aryl and heteroalkyl radicals may be replaced by inert substituents,
R¹, R², R³ and R⁴ are each independently H, straight-chain, branched or cyclic alkyl or alkenyl having from 1 to 18 carbon atoms, aryl or heteroaryl, in which one or more hydrogens in the said alkyl, alkenyl, aryl and heteroalkyl radicals may be replaced by an inert substituent,
or R¹ and R² and/or R³ and R⁴ are joined to one another and form methylene units of a cycloalkane ring —CH₂—(CH₂)ₖ—CH₂— where k=0, 1, 2, 3 or 4,
by reacting a diketene of the formula (II) 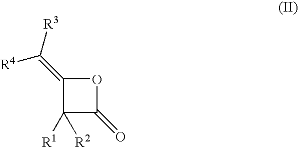
with an active hydrogen-containing compound of the formula ROH, NHRR′ or RSH, in which R and R′ are each as defined above,
which comprises carrying out the reaction continuously in a microreactor.

In the present invention, alkenyl is understood to be an aliphatic carbon radical which has at least one C═C double bond. A plurality of double bonds may be present and may be conjugated.

In the present invention, an inert substituent is understood to be a substituent which is substantially unreactive under the reaction conditions used for the reaction of diketene and the compound containing active hydrogen. Typical examples of inert substituents are alkyls, aralkyls, alkoxy, halogens, in particular F, Cl and Br, —CN, —NO₂, where alkyl and alkoxy radicals are preferably from 1 to 6 carbon atoms and aralkyl is preferably C₆-C₁₀-aryl-C₁-C₆-alkyl, for example including benzyl. In addition, the inert substituent may be a group which would be reactive per se, for instance —OH or —NH, but has been protected by a protecting group.

In the present invention, aryl is understood to be a group which includes at least one aromatic ring. Examples of such aryls are phenyl, sulfophenyl, naphthyl, and further multiring aromatics, for instance pyrene, which may be substituted by inert substituents. Heteroaryls include at least one and optionally a plurality of heteroatoms, for instance N, O, S or/and P, in the aromatic ring structure. Examples of heteroaryls are pyridyl, pyrimidyl, thiazolyl, quinolinyl, indolyl.

In one embodiment of the invention, R¹, R², R³ and R⁴ are each independently H, straight-chain or branched alkyl having from 1 to 18 carbon atoms, typically from 1 to 12 carbon atoms, for example from 1 to 6 carbon atoms. Such alkyls are optionally substituted by inert substituents.

In a particular embodiment of the present invention, R is aryl or heteroaryl, and R′ is H, aryl or heteroaryl. In a preferred embodiment, R is selected from a radical of the following formulae (III), (IV) and (V), and R′ is selected from H or a radical of the following formulae (III), (IV) and (V) 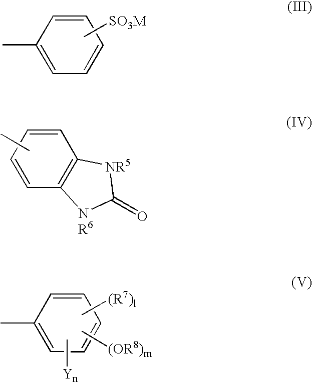
in which
M is hydrogen or an alkali metal, in particular Na or K;
Y is a halogen, in particular Cl,
R⁵ and R⁶ are each independently hydrogen or straight-chain or branched alkyl having from 1 to 6 carbon atoms, in particular methyl or/and ethyl,
R⁷ and R⁸ are each independently straight-chain, branched or cyclic alkyl or alkenyl having from 1 to 18 carbon atoms, in which one or more hydrogens may also be replaced by an inert substituent,
I, m and n are each an integer from 0 to 5, and I+m+n≦5.

In a particular embodiment of the process according to the invention, the corresponding amines are used, i.e. compounds of the formula HNRR′ where R is a compound of the formula (III), (IV) and (V), and R′ is H or a compound of the formula (III), (IV) and (V). R′ is more preferably H.

In a particular embodiment, R is a compound of the formula (IV), R⁵ and R⁶ are each H and R′ is H, i.e. the compound containing active hydrogen is 5-aminobenzimidazolone-2.

In a further embodiment of the present invention, the compound containing active hydrogen is an aliphatic alcohol, i.e. X is O, and R is a straight-chain or branched alkyl which is optionally substituted by inert substituents. In general, aliphatic alcohols having from 1 to 12 carbon atoms, in particular having from 1 to 6 carbon atoms, are used. In particular, the compound containing active hydrogen may be methanol, ethanol, (iso)propanol or tert-butanol.

Particularly preferred products which are prepared by the process according to the invention are methyl 3-oxobutanoate, ethyl 3-oxobutanoate, isopropyl 3-oxobutanoate, isobutyl 3-oxobutanoate, tert-butyl 3-oxobutanoate, 4-acetoacetylaminobenzenesulfonic acid, 5-acetoacetylamino-2-benzimidazolone, acetoacetylaminobenzene, 4-acetoacetamino-1,3-dimethylbenzene, 2-acetoacetylmethoxybenzene, 2-chloroacetoacetaminobenzene, 3-acetoacetamino-4-methoxytoluene-sulfonic acid or a salt thereof.

Particularly preferred examples of β-keto carboxylic acid derivatives are methyl 3-oxobutanoate, isopropyl 3-oxobutanoate, 5-acetoacetylamino-2-benzimidazolone, 4-acetoacetylaminobenzenesulfonic acid or a salt thereof.

Continuously is understood herein to mean that the reactants are fed to the microreactor continuously, in contrast to a batchwise process.

It has been found that the inventive reaction in a microreactor, in comparison to the conventional processes, affords a better yield of the desired reaction product in combination with higher purity, which can be attributed to a lower content of unreacted starting compounds and a lower content of undesired by-products. The higher conversion of starting compounds, in particular of the diketene, also contributes to the avoidance of high contents of or an accumulation of diketene in the reaction mixture, which in practice advantageously constitutes a considerable safety aspect.

Surprisingly, it has also been found that, when the reaction is carried out in a microreactor under comparable reaction conditions, it is possible to use different solvent systems than in the case of reaction in a conventional reactor, for example a tubular reactor. For example, in the reaction of 5-aminobenzimidazolone-2 with diketene to prepare 5-acetoacetylaminobenzimidazolone-2, it has been found that the amine can be used in aqueous solution in the absence of organic solvents (see Example 2). Contrary to this, the known prior art teaches the performance of this reaction in the presence of a water-soluble (C₁-C₄)-alcohol or of a mixture of this alcohol in water (see EP-A-0 648 748). The presence of organic solvents is known to be disadvantageous, in particular from the point of view of potential problems in the product isolation, environmental aspects and costs.

The process according to the invention may be employed even when one of the starting compounds has a relatively poor solubility in aqueous solutions.

The reaction is, if appropriate, carried out in the presence of a catalyst, in particular of a basic catalyst. Suitable catalysts are known to those skilled in the art and are therefore not illustrated in detail herein. For example, the catalyst may be an amine, in particular a tertiary amine, or ammonium salts thereof. For example, sterically hindered tertiary amines are suitable as catalysts. Examples of suitable catalysts are dimethylstearylamine, tributylmethylammonium chloride, NH₄ acetate and 1,4-diazobicyclo[2.2.2]-octane (=DABCO).

Such catalysts are typically present in an amount of from 0.01 to 3 mmol, preferably of from 0.10 to 1.5 mmol and in particular of from 0.25 to 1.0 mmol, per mole of the compound containing active hydrogen.

When the compound containing active hydrogen is an amine, the presence of a catalyst is typically not required, and thus not preferred. However, catalysts are used advantageously when the compound containing active hydrogen is an alcohol or thiol.

It has been found that, when the reaction is carried out in accordance with the invention in a microreactor, no high diketene excess is required, and good yields are nevertheless obtained when the process is at the same time performed in a moderate temperature range.

In a preferred embodiment of the present invention, the molar ratio of diketene (II) to the compound containing active hydrogen is thus from 1:1 to 1.25:1. It has been found that a further reduction in the amount of diketene used is possible in many cases, for example to a ratio of from 1:1 to 1.1:1 or lower, in particular to from 1:1 to 1.05:1.

The temperature of the reaction is appropriately that temperature at which the reaction proceeds with a desired reaction rate and/or selectivity, and at which preferably no thermal decomposition of the reaction product or/and of the starting materials occurs, or/and side reactions are kept at an acceptable degree. At excessively high temperature, thermal decomposition of the reactants or of the desired product may occur, and undesired side reactions may be promoted. At too low a temperature, the reaction may in some cases proceed insufficiently, and the resulting reaction mixture may be contaminated with high contents of unreacted starting materials which may be difficult to remove. It has been found that, in the inventive reaction in a microreactor with a comparable solvent system and comparable or better yields, a lower temperature can be used than in the processes according to the known prior art, which generally has the consequence of a lower content of by-products.

Typically, according to the invention, the reaction is carried out at a temperature of from 40 to 150° C., preferably from 50 to 100° C., in particular at a temperature of from 60 to 80° C.

The pressure at which the inventive reaction is carried out is not particularly critical, and is selected by those skilled in the art as a function of the parameters mentioned above in connection with the temperature. For reasons of cost, it is preferred to carry out the reaction at atmospheric pressure based on the reactor outlet.

The delay time of the components in the microreactor is generally from 1 second to 30 minutes, although longer or shorter delay times are also possible. Typically, the delay times are from 0.5 to 10 minutes, for example from 0.75 to 5 minutes, in particular from 1 to 3 minutes.

The flow rates in the process according to the invention are generally between 0.05 ml/min and 5.0 l/min, more preferably between 0.05 ml/min and 250 ml/min, in particular between 0.1 ml/min and 100 ml/min.

When the diketene and/or the compound containing active hydrogen are in liquid or gaseous form at reaction temperature, they may be fed to the microreactor in substance or in the form of a solution. When they are solid at reaction temperature, they are appropriately fed to the microreactor in the form of a suspension or solution. Suitable diluents and solvents are known to those skilled in the art and are therefore not illustrated in detail. In a preferred embodiment, the diketene or/and the compound containing active hydrogen are fed into the microreactor in the form of an aqueous solution or aqueous suspension.

The microreactors used may, for example, be as disclosed in WO 01/59013 A1.

For example, microreactors as are known from the documents cited there or from publications of the Institut für Mikrotechnik Mainz GmbH, Germany, or else commercially available microreactors, for example the Selecto™ based on Cytos™ from Cellular Process Chemistry GmbH, Frankfurt/Main, may be used.

Microreactors are also understood to be a combination of a static micromixer which contains channels on the microscale as described below, and a heatable delay zone attached thereto, for example a capillary of length from 0.5 to 5 m and an internal diameter between 1 and 5 mm.

The reaction channel of the microreactor used in the present invention is a capillary having any cross section, preferably a round cross section, and generally having a diameter in the longest dimension of from 200 to 1000 μm, preferably from 400 to 800 μm, in particular between 500 to 700 μm.

The advantages of the present invention lie in particular particular in the provision of an efficient process which is advantageous from safety points of view and environmental reasons, and at the same time the enabling of the preparation of very pure products in good yield. For example, based on the preparation of 5-acetoacetylamino-2-benzimidazolone, the content of contaminations by unconverted 5-aminobenzimidazolone-2 in the process according to the invention is typically less than 150 ppm, while this content in conventional processes is in an order of magnitude of up to 500 ppm. The content of contaminations by by-products, for instance acetylacetone, is at the same time typically below 500 ppm when microreactor technology is used, while up to 20 000 ppm are present in the conventional processes.

### EXAMPLES

### Example 1

### Isopropyl 3-oxobutanoate

Isopropanol was admixed with 1,4-diazabicyclo[2.2.2]octane as a catalyst (1 mmol/mol of isopropanol). This solution and diketene were metered by means of two pumps at room temperature into a static micromixer. At the outlet of the micromixer, a stainless steel capillary was attached as a delay zone. The length of the capillary varied between 1.453 and 2.0 m, the internal diameter between 0.19 and 0.3 cm.

The exact metering of the reaction components was controlled gravimetrically. The two reactants were metered in a ratio of 1.0:1.03 (isopropanol/diketene). The flow rate was between 1.0 and 12.5 mol of product/hour or from 2.6 to 31.9 cm³/min, which corresponds to a delay time of from 16 to 3.2 minutes. The delay zone was heated to from 50 to 70° C.

The course of the reaction was monitored by gas chromatography. A reaction mixture having a content of from 95 to 98 area % in the GC of isopropyl 3-oxobutanoate is obtained.

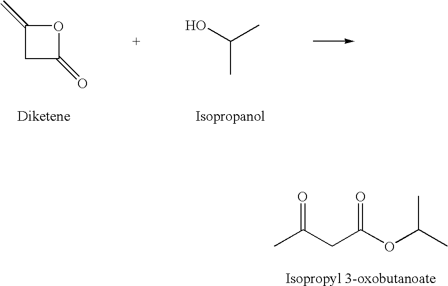

### Example 2

### Methyl 3-oxobutanoate

Methanol was admixed with 1,4-diazabicyclo[2.2.2]octane as a catalyst (1 mmol/mol of methanol). This solution and diketene were metered by means of two toothed-ring micropumps at room temperature into a static micromixer. At the outlet of the micromixer, a stainless steel capillary as a delay zone was attached. The length of the capillary was 1.453 m, the internal diameter 0.19 cm.

The exact metering of the reaction components was controlled gravimetrically. The two reactants were metered in a ratio of 1.0:1.03 (methanol/diketene). The flow rate was 1.0 mol of product/hour or 2.0 cm³/min, corresponding to a delay time of 20.6 minutes. The delay zone was heated to 20° C. The course of the reaction was monitored by gas chromatography. A reaction mixture having a content of 95.0 area % of methyl 3-oxobutanoate is obtained.

### Example 3

### Acetoacetylaminobenzenesulfonic acid

A sulfanilic acid potassium salt solution is prepared batchwise in a conventional manner and adjusted to a concentration of 1.25 M and pH 7.1.

Diketene (13.0 M) is prepared in parallel. After the set reaction parameters of the microreactor (type: Cytos®, Selecto®, CPC) have been attained, the two reactant solutions are conveyed with the aid of the precalibrated pumps into the microreactor. At the outlet of the microreactor, a stainless steel capillary was attached. The length of the capillary varied between 1.453 and 2.0 m, the internal diameter between 0.19 and 0.3 cm.

The exothermic acetoacetylation reaction then takes placed under defined reaction conditions (reaction temperature between 55-75° C., delay time approx. 1-5 minutes) in the microreactor and capillary. The finished product solution is then discharged from the microreactor and collected in a receiver.

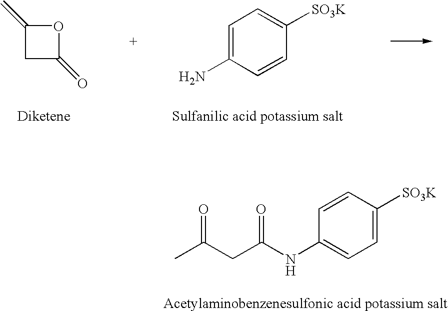

Yield Per Batch (MRT—Microreactor Technology)

| | | | Amount of | | Amount |
|---|---|---|---|---|---|
| | Concentration | Flow rate | substance | | introduced |
| Reactant | [mol/l] | [g/min] | [mmol/min] | [g/min] | [min/1.25 mol] |
| Sulfanilic acid | 1.25 M | 10.0 | 11.26 | 3.5 | 100 |
| potassium | | | | (at 95% | |
| salt solution ¹ | | | | yield) | |
| Diketene | 13.0 M | 1.10 | 13.12 | | |

¹ Density determined using an Areometer: 1.11 g/ml.

MRT can be used to achieve, within 100 min, at a flow rate of, for example, 10.0 ml/min of a 1.25 M sulfanilic acid potassium salt solution, an overall yield of 350 g of acetoacetylsulfanilic acid potassium salt. This corresponds to a yield of 95.0%.

### Example 4

### 5-Acetoacetylamino-2-benzimidazolone (acetolone)

A solution of 5-aminobenzimidazolone (aminolone solution) is prepared batchwise in a conventional manner. 5-Aminobenzimidazolone (aminolone) is introduced into a sodium bisulfite solution heated to 94° C. After activated carbon and ®Dicalite (clarifying assistant) have been added, the aminolone solution is immediately clarified by filtration. The aminolone solution is adjusted to a concentration of 0.5 M. So that aminolone does not crystallize back out of the solution, the 0.5 M aminolone solution is kept at approx. 90° C. In parallel, diketene (13.0 M) is prepared. After the set reaction parameters of the microreactor (type: Cytos/Selecto®, CPC) have been attained, the two reactant solutions are conveyed with the aid of precalibrated pumps into the microreactor. At the outlet of the microreactor, a stainless steel capillary has been attached. The length of the capillary varies between 1.453 and 2.0 m, the internal diameter between 0.19 and 0.3 cm. The exothermic acetoacetylation reaction then takes place under defined reaction conditions (reaction temperature between 55-75° C., delay time approx. 1-5 minutes) in the microreactor and capillary. The reaction mixture is collected in a receiver. After cooling to 20° C., the acetolone precipitates out, so that it can subsequently be filtered off. Serial experiments on the reaction temperature and the delay time show that only above approx. 50° C. and only at a delay time of approx. 60 seconds can a full reaction be achieved.

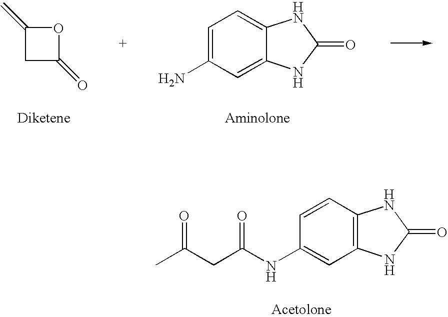

Yield Per Batch (MRT)

| | | | | | Amount |
|---|---|---|---|---|---|
| | | | Amount of | | of acetolone |
| | Concentration | Flow rate | substance | | introduced |
| Reactant | [mol/l] | [g/min] | [mmol/min] | [g/min] | [min/0.2 mol] |
| Aminolone solution | 0.15 M | 36.0 | 5.40 | 1.13 | 37 |
| Diketene | 13.0 M | 0.5 | 6.50 | | |

MRT can be used to achieve, within 60 min, for example at a flow rate of 10.75 ml/min of a 0.15 M aminolone solution, an overall yield of 68.0 g of acetolone. This corresponds to a yield of 90.0%.

1. A process for preparing β-keto carboxylic acid derivative of the formula (I) or a salt thereof 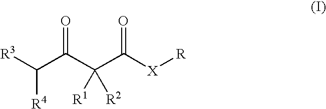
wherein
X is NR′, O or S;
R, R′ are each independently H, straight-chain, branched or cyclic alkyl or alkenyl having from 1 to 18 carbon atoms, aryl or heteroaryl, wherein one or more hydrogens in the alkyl, alkenyl, aryl and heteroalkyl radicals are optionally replaced by an inert substituent,
R¹, R², R³ and R⁴ are each independently H, straight-chain, branched or cyclic alkyl or alkenyl having from 1 to 18 carbon atoms, aryl or heteroaryl, wherein one or more hydrogens in the alkyl, alkenyl, aryl and heteroalkyl radicals are optionally replaced by an inert substituent, or R¹ and R² and/or R³ and R⁴ are joined to one another and form methylene units of a cycloalkane ring —CH₂—(CH₂)ₖ—CH₂— where k=0, 1, 2, 3 or 4,
comprising the step of reacting a diketene of the formula (II) 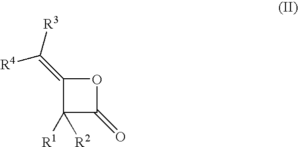
with an active hydrogen-containing compound of the formula ROH, NHRR′ or RSH, and
wherein the reacting step is carried out continuously in a microreactor.

2. The process as claimed in claim 1, wherein R, R′, R¹, R², R³ and R⁴ are each independently H, straight-chain or branched alkyl having from 1 to 6 carbon atoms, phenyl, sulfophenyl, naphthyl, benzyl, pyridyl, pyrimidyl, thiazolyl, quinolinyl or indolyl, each of which is optionally substituted by alkyl, aralkyl, alkoxy, cyano, nitro or halogen.

3. The process as claimed in claim 1 wherein R is a radical of the following formulae (III), (IV) or (V), and R′ is hydrogen or a radical of the following formulae (III), (IV) or (V) 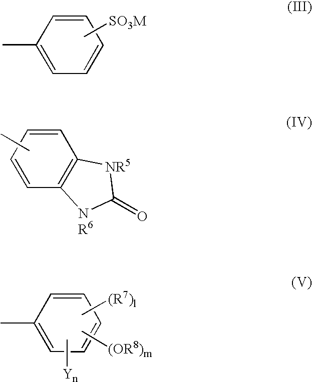
wherein M is hydrogen or an alkali metal;
Y is a halogen,
R⁵ and R⁶ are each independently straight-chain or branched alkyl having from 1 to 6 carbon atoms,
R⁷ and R⁸ are each independently straight-chain, branched or cyclic alkyl or alkenyl having from 1 to 18 carbon atoms, wherein one or more hydrogens of the alkyl or alkenyl are optionally replaced by an inert substituent,
I, m and n are each an integer from 0 to 5, and I+m+n≦5.

4. The process as claimed in claim 1, wherein the β-keto carboxylic acid derivative is selected from the group consisting of methyl 3-oxobutanoate, ethyl 3-oxobutanoate, isopropyl 3-oxobutanoate, isobutyl 3-oxobutanoate, tert-butyl 3-oxobutanoate, 4-acetoacetylaminobenzenesulfonic acid, 5-acetoacetylamino-2-benzimidazolone, acetoacetylaminobenzene, 4-acetoacetamino-1,3-dimethylbenzene, 2-acetoacetylmethoxybenzene, 2-chloroacetoacetaminobenzene, 3-acetoacetamino-4-methoxytoluene-6-sulfonic acid and a salt thereof.

5. The process as claimed in claim 1, wherein the reacting step is carried out in the presence of a basic catalyst.

6. The process as claimed in claim 1, wherein the molar ratio of the diketene of formula (II) to the active hydrogen-containing compound is from 1:1 to 1.25:1.

7. The process as claimed in claim 1, wherein the reacting step is carried out at a temperature of from 40 to 150° C.

8. The process as claimed in claim 1, wherein the reacting step is carried out with a delay time of the components in the microreactor of from 1 second to 30 minutes.

9. The process according to claim 1, wherein the reacting step further comprises feedin the active hydrogen-containing compound into the microreactor as an aqueous suspension or aqueous solution.

10. The process as claimed in claim 1, wherein the microreactor consists of a static micromixer and a heatable delay zone.

11. The process as claimed in claim 1, wherein the microreactor comprises a static micromixer and a heatable delay zone.

12. A β-keto carboxylic acid derivative or a salt thereof made in accordance with the process of claim 1.
